(19) United States
(12) Patent Application Publication
(10) Pub. No.: US 2024/0157294 A1
(43) Pub. Date: May 16, 2024

(54) COMPACT, MICROCOMPOSITE GAS FILTER

(71) Applicant: QUALCOMM Technologies, Inc., San Diego, CA (US)

(72) Inventors: Eric Paul LEE, Los Altos, CA (US); Jim Chih-Min CHENG, Fremont, CA (US)

(21) Appl. No.: 18/508,934

(22) Filed: Nov. 14, 2023

Related U.S. Application Data
(60) Provisional application No. 63/383,957, filed on Nov. 16, 2022.

(51) Int. Cl. B01D 53/86; B01D 39/20; B01D 46/00; B01D 53/85; G01N 33/00

(52) U.S. Cl. CPC B01D 53/86; B01D 39/20; B01D 46/0036; B01D 53/85; G01N 33/0013

(57) ABSTRACT

Commercially available gas filters are too large for mobile form-factor gas sensors. These sensors require filters with planar dimensions in the millimeter range and thicknesses of 1 mm or less and combined with long term and harsh conditions survivability. Furthermore, custom filters made small enough to be applied onto mobile form-factor gas sensors are generally not specific enough to support multi-gas detection. This invention describes a novel structured filter: (1) derived from principles of macroscale gas separation columns, (2) made compact enough to be utilized at millimeter scale and microscale levels, and (3) made compatible with mobile requirements and some methodology for usage in micro gas spectroscopy platforms.

### CROSS-REFERENCE TO RELATED APPLICATION

This Patent Application claims priority to Provisional Patent Application No. 63/383, 957, filed on Nov. 16, 2022, entitled “COMPACT, MICROCOMPOSITE GAS FILTER,” and assigned to the assignee hereof. The disclosure of the prior Application is considered part of and is incorporated by reference into this Patent Application.

### FIELD OF THE DISCLOSURE

Aspects of the present disclosure generally relate to a gas filter and, for example, to gas filtration in space-constrained platforms requiring high selectivity.

### BACKGROUND

Gas sensors all suffer from limited selectivity in many applications. Whether the basis of the gas sensing is optical, electrical, or electrochemical, the signal attained from the gas sensor is both a function of the amount of the target gas species present, and the unwanted signal generated by other gaseous species in the sample mix to which the sensing element is sensitive.

The signal from these other gaseous species is likely to be convoluted with the target gas signal in the case that the physical-chemical properties of the unwanted gases is similar to that of the target gas; the gas sensor is said to have “cross sensitivity” to these other gas species in this case.

So as to reduce the cross-sensitivity of gas sensors to unwanted gasses, filters are often added to the sensing element so as to separate the cross-sensitive gas species. For example, mass spectroscopy is not able to separate nitrogen (N₂) and carbon monoxide (CO) gas signals since the two gases have the same molecular weight. In addition, structurally similar molecules such as ethanol and methanol have the same functional hydroxyl (—OH) group and ethylene (CH₂) backbone, which will make their spectral fingerprint very similar

Absorptive materials are a very commonly used form of filter for gas sensors. Absorptive materials are limiting because they will saturate and therefore require large volumes to effectively remove undesirable gas species; for absorptive filter materials, incorporation into a small form factor becomes almost impossible. Saturation is not the only hurdle for gas filtration, another limitation is off-gassing when the sensor is no longer exposed to a gaseous sample. Over time the absorbed gas species will off-gas into the sensor and give a false positive reading. Furthermore, some gas molecules may irreversibly adsorb onto the surface of the filter, limiting the filter's lifetime.

Catalytic filters are another useful class of filtering medium. However, catalysts have limited selective capabilities because they only target chemical functional groups. For example, a catalyst designed to filter out alcohols will not necessarily be able to selectively filter methanol from ethanol or isopropanol. In addition, catalysts are not capable of filtering inert gaseous species or filtering based on molecular size. Therefore, non-reactive and small gas molecules will not be filtered.

Acting similarly to catalytic filtration, dehydrated enzymes can be used for gas-phase filtration. Such enzymes allow for complete and selective conversions of specific gas species to known products. However, enzymatic activity and stability is highly influenced by water content, making enzymes unreliable under different humidities and temperatures.

Thus one of the most basic, yet powerful of filtration mechanisms remains size exclusion. For gases, this requires the pore sizes to be on the order of a single angstrom to at maximum several nanometers in size. The materials are typically known as molecular sieves and have several known issues from absorption of materials on their surface such as moisture to blockage of their pores.

In the manufacture of macroscale filters, gas path length can be extended by thickening the filters to help block various gaseous species. Additionally, the addition of multiple types of materials to the filters for filtration is a more straightforward manufacturing problem. For mobile applications where there is very limited space for the filter, thicknesses of 1 millimeter (mm) or less are required which require more advanced planar processing and careful selection of filter materials.

An example of molecule sieves are zeolites, which are microporous, aluminosilicate minerals mainly including silicon (Si), aluminum (Al), oxygen (O), and metals including titanium (Ti), tin (Sn), zinc (Zn), or the like. Due to a highly regular pore structure of molecular dimension, zeolite crystal structures allow their ability to selectively sort molecules based primarily on size exclusion. These minerals are also commonly used as catalysts and absorbents. Zeolites can accommodate a wide variety of cations, such as sodium cations (Na+), potassium cations (K+), calcium cations (Ca₂₊), magnesium cations (Mg₂₊), or the like, allowing for several different acidities. This enables zeolites to catalyze several acid reactions, as well as to capture specific ions while allowing others to pass through. In addition, their high heat of adsorption and capability to hydrate and dehydrate while maintaining structural stability makes them prime candidates as commercial adsorbents and catalysts.

Zeolitic imidazolate frameworks (ZIFs) are a class of metal-organic frameworks which also have “molecular sieve” capabilities. ZIFs are composed of tetrahedrally-coordinated transition metal ions (e.g., iron (Fe), cobalt (Co), copper (Cu), Zn) connected by imidazolate linkers and have a similar crystal structure as zeolites. ZIFs have been used for filtering applications such as carbon capture.

In commercial use, molecular sieve materials are commonly used in powder form and packed into columns or traps to increase the gas path length or are added directly in with reacting reagents. These processes require no support material or binder, which makes manufacture of molecular sieve filters on a macro-scale challenging.

In the manufacture of macro-scale filters based on zeolites and ZIFs, gas path length can be extended by thickening the filters to help block various gaseous species. In some embodiments, a combination of multiple types of zeolites and ZIFs to the filters can be used. For mobile applications where there is very limited space for the filter, thicknesses of 1 mm or less are required, which require more advanced planar processing and careful selection of filter materials.

Additionally, the structure holding the material needs to be carefully processed to ensure robustness over a several year lifetime of the filter itself. Current state of the art filters are not made in these form factors and many of active filtration materials are not viable for these form factors.

### SUMMARY

In some implementations, a gas filter includes a microcomposite structure comprising: a filter medium comprising an active material, having a combination of size exclusive, absorptive, and catalytic properties, that enables gaseous molecules below a certain size and of a particular chemical nature not to be absorbed or reacted to pass through the active material, wherein the active material comprises a molecular sieve, and wherein a planar dimension of the filter medium is less than or equal to approximately 1 centimeter (cm) and a thickness of the filter medium is less than or equal to approximately 2 millimeters (mm).

In some implementations, a gas filter includes a microcomposite structure comprising: a filter medium comprising an active material having at least one of a size exclusive property, an absorptive property, or a catalytic property, wherein a planar dimension of the filter medium is less than or equal to approximately 1 cm and a thickness of the filter medium is less than or equal to approximately 2 mm.

In some implementations, a gas sensor system includes a gas filter comprising: a filter medium that enables gaseous molecules below a certain size and of a particular chemical nature not to be absorbed or reacted to pass through a molecular sieve of the filter medium, wherein a planar dimension of the filter medium is less than or equal to approximately 1 cm and a thickness of the filter medium is less than or equal to approximately 2 mm, and wherein the molecular sieve incorporates particulate material having a size in range from approximately 100 nanometers (nm) to 500 micrometers (μm), wherein the particulate material has microporous, mesoporous or macroporous nature.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the scope of the appended claims. Characteristics of the concepts disclosed herein, both their organization and method of operation, together with associated advantages will be better understood from the following description when considered in connection with the accompanying figures. Each of the figures is provided for the purposes of illustration and description, and not as a definition of the limits of the claims.

### BRIEF DESCRIPTION OF THE DRAWINGS

So that the above-recited features of the present disclosure can be understood in detail, a more particular description, briefly summarized above, may be had by reference to aspects, some of which are illustrated in the appended drawings. It is to be noted, however, that the appended drawings illustrate only certain typical aspects of this disclosure and are therefore not to be considered limiting of its scope, for the description may admit to other equally effective aspects. The same reference numbers in different drawings may identify the same or similar elements.

FIG. 1 depicts a conventional filter medium comprising a pressed powder such as a molecular sieve. Due to the poor cohesive strength of such powders, a percolation path or crack in the medium can easily be formed through which gas can leak unfiltered. In this case, the efficacy of the filter becomes impaired.

FIG. 2 is an illustration of how binder particles works with an active material that has pores to ensure activity to target gases.

FIG. 3 depicts a filter medium comprising a pressed powder such as a molecular sieve. In FIG. 3, a binder is added to the powder prior to pressing so as to bind the powder together and provide additional cohesive strength to the element. An optional hydrophobic and/or oleophobic layer may be coated on one or both sides of the filter medium.

FIG. 4 depicts a gas filter comprising a first filter medium pressed powder such as a molecular sieve. An optional a binder is added to the powder prior to pressing. The gas filter further comprises a superficial breathable fluoropolymer layer (e.g., polytetrafluoroethylene (PTFE), expanded PTFE, (ePTFE,), polyvinyl fluoride (PVF), hydrophobic polyethersulfone (PES), or the like) laminated to the first filter medium so as to provide waterproofing, protection from oils, and/or physical protection from scratches. The gas filter has optional impermeable side walls forcing the flow or diffusion of gas to pass through both filter media.

FIG. 5 depicts the filter structure of FIG. 4 augmented with an additional filter medium such as an absorptive filter, a catalytic filter, or an enzyme so as to provide additional filtering over and above that provided by the first filter medium. Multiple layers of additional filter media can be added to tailor and/or augment the filtering capability. The order of the layers can be varied, depending on the application.

FIG. 6 depicts a filter in combination with an electrochemical cell and a drive circuit. In this configuration a multi-point bias operation of the electrochemical cell/ drive circuit can further down-select any unfiltered gas and identify target species by enhancing or suppressing different reactions.

FIG. 7 shows Fourier transform infrared (FTIR) graphs of zeolite performance against several different gases.

FIG. 8 shows FTIR graphs of zeolitic imidazolate framework (ZIF) and other absorber performance against several different gases.

### DETAILED DESCRIPTION

Various aspects of the disclosure are described more fully hereinafter with reference to the accompanying drawings. This disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. One skilled in the art should appreciate that the scope of the disclosure is intended to cover any aspect of the disclosure disclosed herein, whether implemented independently of or combined with any other aspect of the disclosure. For example, an apparatus may be implemented or a method may be practiced using any number of the aspects set forth herein. In addition, the scope of the disclosure is intended to cover such an apparatus or method which is practiced using other structure, functionality, or structure and functionality in addition to or other than the various aspects of the disclosure set forth herein. It should be understood that any aspect of the disclosure disclosed herein may be embodied by one or more elements of a claim

Creation of an effective gas filter for an application in which space is constrained, such as a mobile application, requires: (1) a combination of materials capable of actively blocking, absorbing and/or catalyzing an input gas so only to pass target analytes; (2) a composite micro and nanostructure formed from active materials and potentially binding materials which results in sufficient porosity for sufficiently rapid diffusion of gas through the filter, while keeping target gases from passing through; (3) a layered structure to protect against condensing moisture (e.g., to make the filter water resistant and ideally capable of achieving a desired requirement (e.g., Ingress Protection (IP) code 67 or 68)); (4) morphology and adhesive structure to attach to an underlying sensor or other electronic device which requires such filtration (e.g., a hard disk drive); (5) thin yet robust construct as to allow for handling associated with mobile device applications (e.g., resistance to vibration, drops, pressure variations, or temperature variations).

What makes a filter module described herein novel is not just the parts and processing required to realize the filter module, but the integrated parts which work complementary to achieve the proper amount of filtration.

A filtering medium enabling the passing of gaseous molecules below a certain size can be made from a material such as a molecular sieve or a zeolite. Molecular sieves, and a variant called metal organic frameworks (MOFs), comprise crystalline substances (e.g., a zeolite) with pores of molecular dimensions that permit the passage of molecules below a certain size. In contrast to an absorptive filter, the molecular sieve is capable of enduring extended exposure to high concentrations of gasses without becoming saturated. However, the pores of the molecular sieves can still be blocked depending on which gases the molecular sieve has to filter. Additionally, with MOF structures, catalytic elements can be incorporated into the structure to better target specific gas species (in addition to size exclusion). Molecular sieve materials like zeolites have some capability for this as well depending on charge based on incorporated ions. Accordingly, gas filters based on molecular sieves can be made with extremely small form factor and, hence, are ideal filters for integration with small form-factor gas sensors used in mobile applications and other space-constrained applications. Notably, filters with thickness of less than or equal to approximately 2 mm can be produced.

The active filter medium may be comprised of layers of active material to work in tandem to filter out interfering gas species. In one embodiment, the first layer of the filter medium is a size exclusion material which can come in particulate or membrane form. If in particulate form, the particles of the filter medium can be bound together with a binder (e.g., an inert and hydrophobic binder). Additionally, or alternatively, the particles of the filter medium can be bound into a substrate to increase robustness of the filter medium and make the filter medium easier to handle. In some embodiments, the size exclusion material can be functionalized with specific ions and functional groups to help reject certain charged gas species. Functional groups with specific terminal ends can also help to temporarily bind gases to their surface to help with rejection of certain gas molecules. In some embodiments, a catalyst can be included as a second layer to make a stack (e.g., similar to a larger, gas chromatography column). Shrinking the longitudinal path of a chromatography column into a thin, planar, but still multi-layered structure is part of the novelty of this solution.

In some embodiments, to provide efficient and effective means of filtration, all gas incident on the filter medium should be made to pass through the molecular sieve. Therefore, no percolation paths should exist between front and back sides of the filter medium through which gas can pass without also passing through the molecular sieve. Similarly, no percolation paths should exist which are sufficiently tortuous and result in hindering direct diffusion of molecules.

Molecular sieve materials are typically formed as powders. Therefore, in order to be used as a filter medium, the sieve powder can be pressed and/or sintered so as to eliminate percolation paths between powder grains. Alternatively, the sieve may be formed as a single crystal.

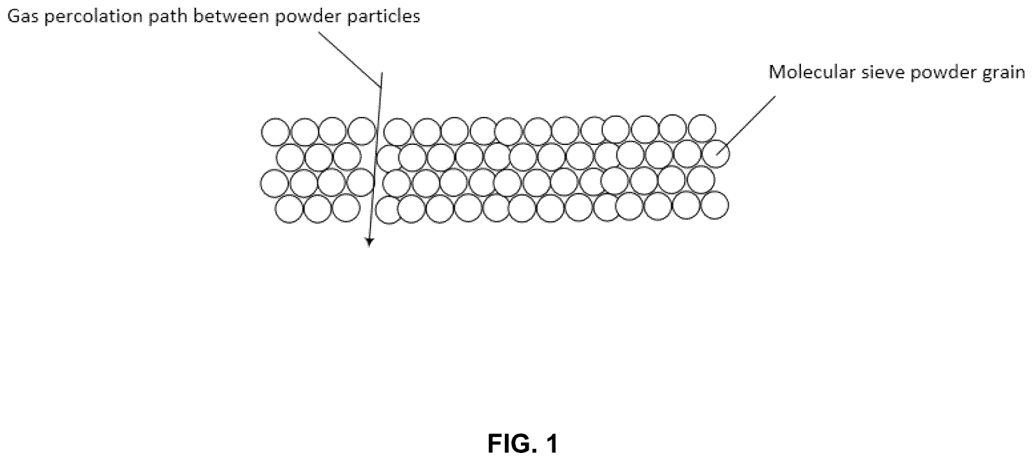

Conventionally, pressed powdered materials have poor tensile strength and break easily under tensile stress, bending, or scratching, as shown in FIG. 1. Accordingly, a practical filter medium comprising a molecular sieve requires strengthening. This may be achieved by sintering the powder together, or by incorporating a binder so as to bind the powder particles together without blocking pores of the molecular sieve. In some embodiments, the binder may fill-in and block percolation paths between powder particles. In some embodiments, the binder may be a low-outgassing polymer or adhesive such as a polyurethane in powdered form, fiber form, or another form.

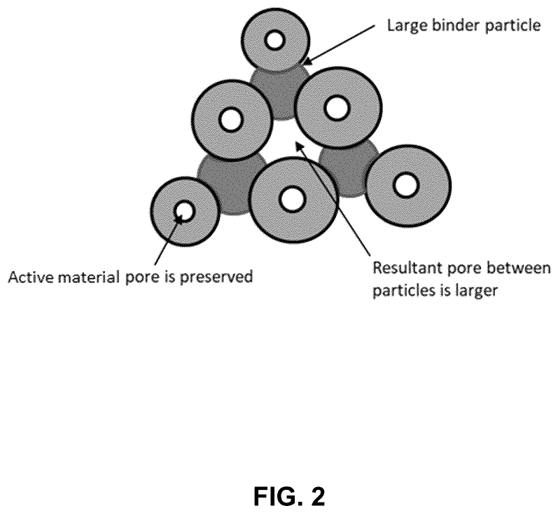

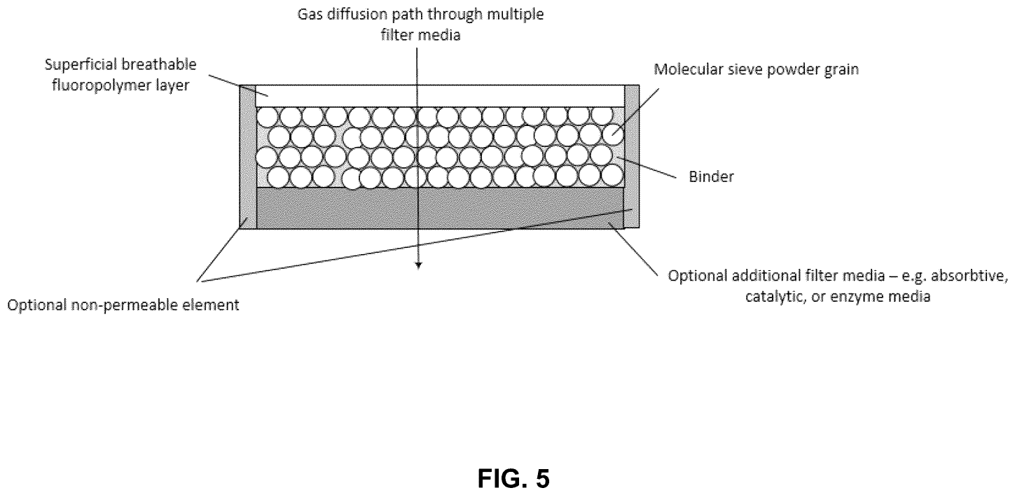

In some embodiments, the binder may be selected (e.g., ceramic, polymer, or the like) so that the binder does not occlude activity of the active material (as mentioned above), such as a zeolite which has a catalytic surface which catalyzes certain target gases. Pore size achieved in such structures control a diffusion rate and can be used to selectively separate and block certain gases due to the different levels of activity gases may have going through these pores, and the gas molecule size. In one example, CO which is a less reactive, inorganic gas with a small size, is able to pass through longer, narrow pathlengths without significant loss, while volatile organic compounds (VOCs) such as ethanol require larger pore sizes and shorter pathlengths to minimize interaction with the active materials (e.g., zeolite) so as not to impede diffusion. FIG. 2 is an illustration of how particles of a binder work with an active material (e.g., a zeolite) that comprises pores to ensure activity to target gases. As shown in FIG. 5, the binder may in some embodiments comprise particles that do not block pores of the active material, meaning that activity to target gases provided by the pores in the active material is preserved.

In some embodiments, the binder choice can also controls strength of the structure for long term reliability and handling. Novelty in this microcomposite structure is the ability to achieve microscale pores (e.g., on the order of 1 μm-300 μm in scale), while integrating in high density (e.g., greater than 30% weight loading) of active material like zeolite with greater than on average 50% of its surface area still functional and/or micro/nanoscale porosity non-occluded. In some aspects, such pore and gas pathlengths can be achieved via the binder and structural filler or by compacting the overall structure in a controlled manner so as to narrow or occlude excess gas pathways and, overall, achieve a thinner form factor.

In addition, the structure may be able to withstand a medium shear force exceeding 0.5 kilograms (kg) or may be pliable, but may still be robust enough to temporarily deform so as not to break or tear, while still returning to an original form (or sufficiently close to the original form) to retain filter performance (e.g., gas diffusion, absorption/catalytic activity).

In some embodiments, the filter medium may be rendered partially or fully hydrophobic and/or oleophobic by the inclusion of hydrophobic and/or oleophobic elements. For example, a molecular sieve powder may be mixed with a hydrophobic powder such as a powdered fluorinated polymer, and a filter medium may be formed from the mixture. In some cases, the binding medium may provide a degree of hydrophobicity or oleophobicity. For example, molecular sieve powders bound with a fluorinated polymer such as PTFE, can be made to be intrinsically hydrophobic.

Additionally, or alternatively, the molecular-sieve-based filter medium may be coated with a hydrophobic and/or oleophobic coating after formation.

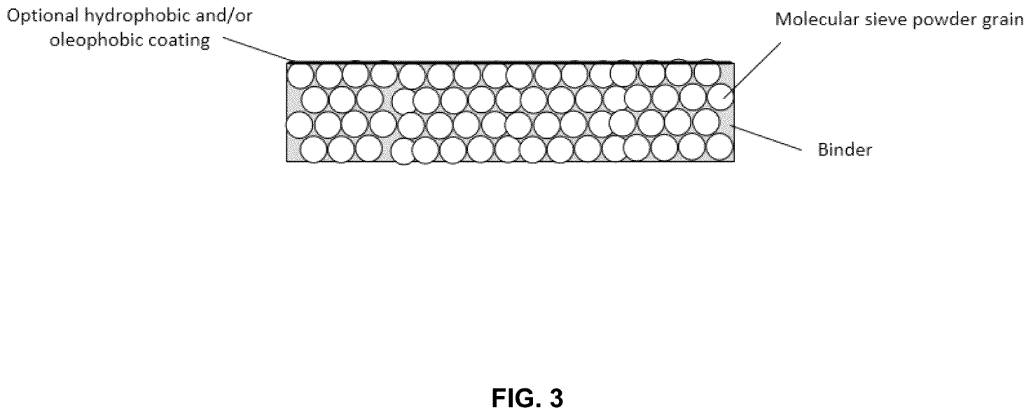

In some embodiments, depending on the choice of molecular sieve material, the filter medium may be able to reject materials from passing based on electronic charge of the materials and/or polarity of the materials, in addition to being able to reject materials from passing based on size. An example of such a structure is shown in FIG. 3.

In some embodiments, the filter medium may be paired with one or more additional, layered filter media. For example, a superficial layer of breathable PTFE, or other appropriate breathable fluoropolymer may be placed on a first filter medium in order to provide waterproofing. Example breathable fluoropolymer membranes include Donaldson EN0701957 and Nitto Temish, among other examples.

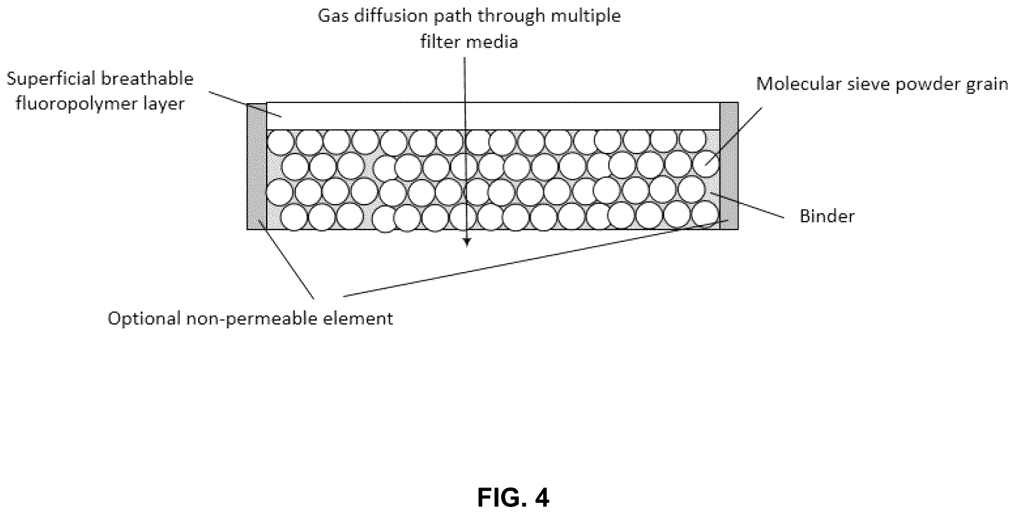

In some embodiments, the filter may comprise stacked materials, laminated with a patterned or breathable adhesive. In some embodiments, an outer edge of the filter stack may be bound with a non-permeable member, thereby blocking gas from penetrating into the filter from the sides, and forcing gas to penetrate the filter assembly through both filter media. An example of such a structure is shown in FIG. 4.

In some embodiments, the filtering medium (e.g., as shown in FIG. 3) may be paired, for example, or layered with one or more filter media comprising catalytic materials, absorbent materials, or enzymes so as to provide additional gas filtration based on chemical structure. The order of the filter media may be selected so as to provide optimal performance. For example, by placing an absorbent media layer after the molecular sieve, the absorbent material is exposed to fewer gasses and is hence less likely to become saturated.

Examples of catalytic materials for uses in such a filter include metallic and organo-metallic particles (e.g., Zif67-A, Zif67-B, and HKUS T-1) or zeolite catalysts (e.g., ZSM-5, ZSM-35, SSZ-13), among other examples.

Examples of absorbent materials include activated carbon, porous polymers (e.g., HaySep D), mesoporous silica, zeolites (e.g., ZSM-35), molecular-sieve activated carbon, or Carboxen 1003, among other examples.

In some embodiments, the catalytic or absorbent materials may be embedded in a matrix such as a sintered ceramic, fused polymer particles, a coated glass fiber (e.g., Porex BioDesign Glass fiber, Fiber Glass 01543 or 00543), or a fabric support (e.g., Calgon).

Examples of filter media able to serve as exclusion materials based on size comprise molecular sieves or zeolites, among other examples.

To reduce or eliminate an impact of adhesives on a gas sensor system, an adhesive used in an assembly of filters may comprise a low-outgassing adhesive or an adhesive outgassing species to which the gas sensor is not sensitive. Example low outgassing adhesives include Nitto 5302A, Nitto 5000NS, 3M 300LSE, or Donaldson EN-DC 07.03.249, among other examples. An example of such a structure is shown in FIG. 5.

In some embodiments, in addition to being constructed in a layered fashion, the multiple filter media types may be formed as a single element. For example, a powdered molecular sieve may be mixed with a powdered catalytic, absorptive, enzyme media, and an appropriate binder and may be pressed. In one embodiment, multiple filter media are mixed together with a binder and are thermally pressed between two breathable fluoropolymer layers to bind a filter stack together to form one piece.

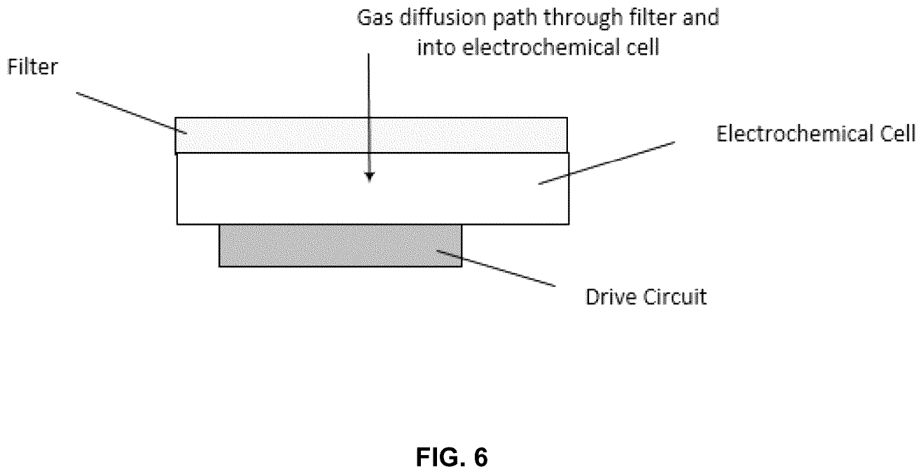

In some embodiments, to provide reduced cross-sensitivity, a gas sensor system comprising a sensor capable of selectivity may be integrated with a gas filter described herein. An example of such an assembly is shown in FIG. 6. Gas selectivity based on the combined use of a catalytic, size exclusion filter and multipoint bias operation of the electrochemical cell is capable of correctly measuring gas signals, along with accurately measuring ambient humidity and temperature. Such a system can be integrated in an array of products, such as mobile devices or Internet-of-things (IOT) environmental monitoring stations, among other examples.

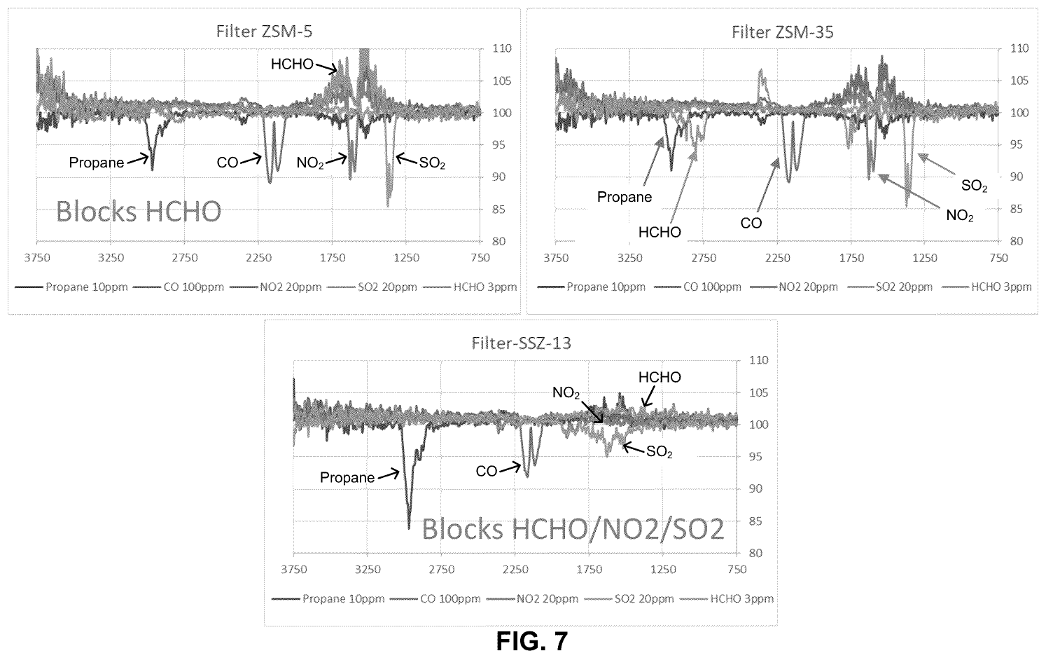

In one example embodiment, a gas filter assembled by hot pressing a zeolite material into a cellulose matrix resulted in selective filtering or gases. FIG. 7 shows FTIR spectrographs after passing multiple gases through filters made with different zeolites. The diagram in the top left of FIG. 7 corresponds to a filter assembled with ZSM-5. In this example, propane, CO, nitrogen dioxide (NO₂), and sulfur dioxide (SO₂) pass through the filter, but formaldehyde (HCHO) is blocked. As shown in the top right diagram of FIG. 7, in the case of ZSM-35, propane, CO, NO₂, SO₂, and formaldehyde pass through the filter. As shown in the bottom diagram of FIG. 7, in the case of an SSZ-13, propane and CO pass through, but NO₂, SO₂ and formaldehyde are blocked.

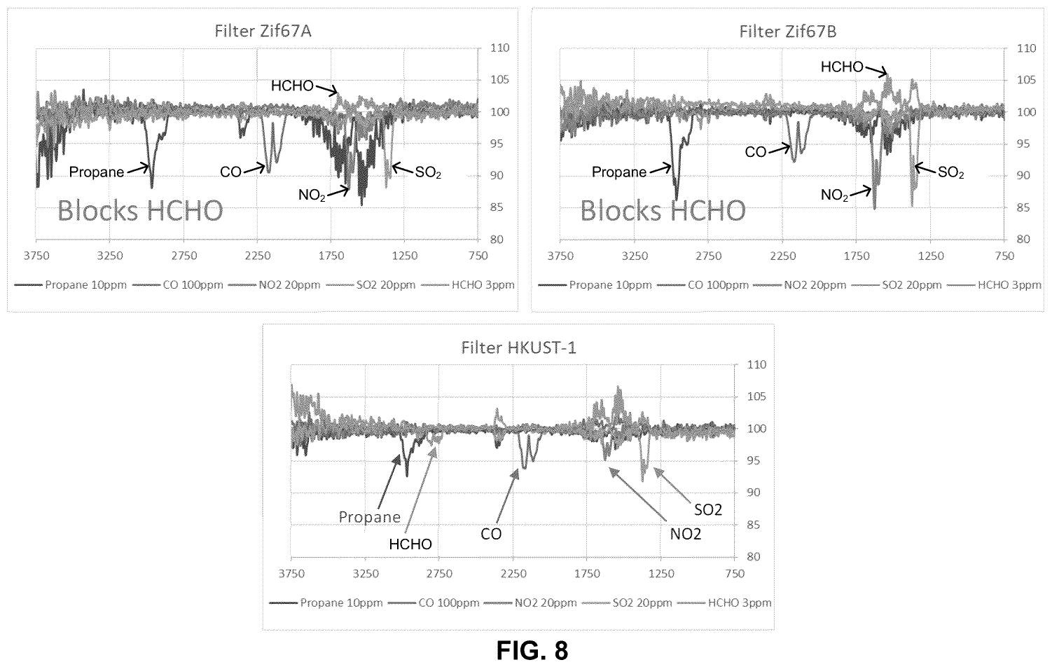

In another example embodiment, filters assembled by hot pressing MOFs into a cellulose matrix resulted in selective filtering of gases. FIG. 8 shows FTIR spectrographs after passing multiple gases through filters made with different MOFs. As shown in the top left and top right diagrams of FIG. 8, filters assembled with ZIF67-A and ZIF67-B allow propane, CO, NO₂, and SO₂ through, but block formaldehyde. As shown in the bottom diagram of FIG. 8, in the case of HKUST-1, propane, CO, NO₂, SO₂, and formaldehyde pass through.

The following provides an overview of some Aspects of the present disclosure:

Aspect 1: A gas filter, comprising: a microcomposite structure comprising: a filter medium comprising an active material, having a combination of size exclusive, absorptive, and catalytic properties, that enables gaseous molecules below a certain size and of a particular chemical nature not to be absorbed or reacted to pass through the active material, wherein the active material comprises a molecular sieve, and wherein a planar dimension of the filter medium is less than or equal to approximately 1 centimeter (cm) and a thickness of the filter medium is less than or equal to approximately 2 millimeters (mm).

Aspect 2: The gas filter of Aspect 1, wherein the active material incorporates particulate material having a size in range from approximately 100 nanometers (nm) to 500 micrometers (μm), wherein the particulate material has microporous, mesoporous or macroporous nature.

Aspect 3: The gas filter of Aspect 2, wherein the particulate material has a nanostructure associated with capturing or catalyzing reactions of one or more particular gases.

Aspect 4: The gas filter of Aspect 2, wherein the particulate material is pressed or sintered to reduce or eliminate percolation paths between grains of the particulate material.

Aspect 5: The gas filter of Aspect 2, wherein the filter medium further comprises a binder to bind grains of the particulate material together while maintaining porosity or permeability to allow one or more particular gases to pass through the gas filter.

Aspect 6: The gas filter of Aspect 5, wherein the binder fills-in or at least partially blocks percolation paths between the grains of the particulate material.

Aspect 7: The gas filter of Aspect 5, wherein the binder comprises a low-outgassing polymer or adhesive.

Aspect 8: The gas filter of Aspect 5, wherein the binder comprises a hydrophobic material.

Aspect 9: The gas filter of Aspect 5, wherein the binder comprises an oleophobic material.

Aspect 10: The gas filter of Aspect 2, wherein the particulate material is bound in a scaffold that is compacted in association with controlling percolation pathways and gas diffusion path and length to provide selectivity between gases. This results in thinner filter / filter stack fitting more compact applications.

Aspect 11: The gas filter of any of Aspects 1-10, wherein the active material is functionalized with a particular ion or a particular functional group in association with rejecting a particular gas species.

Aspect 12: The gas filter of any of Aspects 1-11, further comprising a hydrophobic layer on a surface of the filter medium.

Aspect 13: The gas filter of any of Aspects 1-12, further comprising an oleophobic layer on a surface of the filter medium.

Aspect 14: The gas filter of any of Aspects 1-13, further comprising a superficial breathable fluoropolymer layer on a surface of the filter medium.

Aspect 15: The gas filter of any of Aspects 1-14, further comprising an impermeable layer on a sidewall of the filter medium.

Aspect 16: The gas filter of any of Aspects 1-15, wherein the gas filter further comprises another filter medium that includes an enzyme.

Aspect 17: The gas filter of any of Aspects 1-16, wherein the gas filter is included in a gas sensing system comprising a gas detector and a drive circuit.

Aspect 18: A gas filter, comprising: a microcomposite structure comprising: a filter medium comprising an active material having at least one of a size exclusive property, an absorptive property, or a catalytic property, wherein a planar dimension of the filter medium is less than or equal to approximately 1 centimeter (cm) and a thickness of the filter medium is less than or equal to approximately 2 millimeters (mm).

Aspect 19: The gas filter of Aspect 18, wherein the active material incorporates particulate material having a size in range from approximately 100 nanometers (nm) to 500 micrometers (μm), wherein the particulate material has microporous, mesoporous or macroporous nature.

Aspect 20: A gas sensor system, comprising: a gas filter comprising: a filter medium that enables gaseous molecules below a certain size and of a particular chemical nature not to be absorbed or reacted to pass through a molecular sieve of the filter medium, wherein a planar dimension of the filter medium is less than or equal to approximately 1 centimeter (cm) and a thickness of the filter medium is less than or equal to approximately 2 millimeters (mm), and wherein the molecular sieve incorporates particulate material having a size in range from approximately 100 nanometers (nm) to 500 micrometers (μm), wherein the particulate material has microporous, mesoporous or macroporous nature.

The foregoing disclosure provides illustration and description but is not intended to be exhaustive or to limit the aspects to the precise forms disclosed. Modifications and variations may be made in light of the above disclosure or may be acquired from practice of the aspects.

Even though particular combinations of features are recited in the claims and/or disclosed in the specification, these combinations are not intended to limit the disclosure of various aspects. Many of these features may be combined in ways not specifically recited in the claims and/or disclosed in the specification. The disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a, b, c, a+b, a+c, b+c, and a+b+c, as well as any combination with multiples of the same element (e.g., a+a, a+a+a, a+a+b, a+a+c, a+b+b, a+c+c, b+b, b+b+b, b+b+c, c+c, and c+c+c, or any other ordering of a, b, and c).

No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the terms “set” and “group” are intended to include one or more items and may be used interchangeably with “one or more.” Where only one item is intended, the phrase “only one” or similar language is used. Also, as used herein, the terms “has,” “have,” “having,” or the like are intended to be open-ended terms that do not limit an element that they modify (e.g., an element “having” A may also have B). Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A gas filter, comprising:
a microcomposite structure comprising:
a filter medium comprising an active material, having a combination of size exclusive, absorptive, and catalytic properties, that enables gaseous molecules below a certain size and of a particular chemical nature not to be absorbed or reacted to pass through the active material,
wherein the active material comprises a molecular sieve, and
wherein a planar dimension of the filter medium is less than or equal to approximately 1 centimeter (cm) and a thickness of the filter medium is less than or equal to approximately 2 millimeters (mm).

2. The gas filter of claim 1, wherein the active material incorporates particulate material having a size in range from approximately 100 nanometers (nm) to 500 micrometers (μm), wherein the particulate material has microporous, mesoporous or macroporous nature.

3. The gas filter of claim 2, wherein the particulate material has a nanostructure associated with capturing or catalyzing reactions of one or more particular gases.

4. The gas filter of claim 2, wherein the particulate material is pressed or sintered to reduce or eliminate percolation paths between grains of the particulate material.

5. The gas filter of claim 2, wherein the filter medium further comprises a binder to bind grains of the particulate material together while maintaining porosity or permeability to allow one or more particular gases to pass through the gas filter.

6. The gas filter of claim 5, wherein the binder fills-in or at least partially blocks percolation paths between the grains of the particulate material.

7. The gas filter of claim 5, wherein the binder comprises a low-outgassing polymer or adhesive.

8. The gas filter of claim 5, wherein the binder comprises a hydrophobic material.

9. The gas filter of claim 5, wherein the binder comprises an oleophobic material.

10. The gas filter of claim 2, wherein the particulate material is bound in a scaffold that is compacted in association with controlling percolation pathways and gas diffusion path and length to provide selectivity between gases.

11. The gas filter of claim 1, wherein the active material is functionalized with a particular ion or a particular functional group in association with rejecting a particular gas species.

12. The gas filter of claim 1, further comprising a hydrophobic layer on a surface of the filter medium.

13. The gas filter of claim 1, further comprising an oleophobic layer on a surface of the filter medium.

14. The gas filter of claim 1, further comprising a superficial breathable fluoropolymer layer on a surface of the filter medium.

15. The gas filter of claim 1, further comprising an impermeable layer on a sidewall of the filter medium.

16. The gas filter of claim 1, wherein the gas filter further comprises another filter medium that includes an enzyme.

17. The gas filter of claim 1, wherein the gas filter is included in a gas sensing system comprising a gas detector and a drive circuit.

18. A gas filter, comprising:
a microcomposite structure comprising:
a filter medium comprising an active material having at least one of a size exclusive property, an absorptive property, or a catalytic property,
wherein a planar dimension of the filter medium is less than or equal to approximately 1 centimeter (cm) and a thickness of the filter medium is less than or equal to approximately 2 millimeters (mm).

19. The gas filter of claim 18, wherein the active material incorporates particulate material having a size in range from approximately 100 nanometers (nm) to 500 micrometers (μm), wherein the particulate material has microporous, mesoporous or macroporous nature.

20. A gas sensor system, comprising:
a gas filter comprising:
a filter medium that enables gaseous molecules below a certain size and of a particular chemical nature not to be absorbed or reacted to pass through a molecular sieve of the filter medium,
wherein a planar dimension of the filter medium is less than or equal to approximately 1 centimeter (cm) and a thickness of the filter medium is less than or equal to approximately 2 millimeters (mm), and
wherein the molecular sieve incorporates particulate material having a size in range from approximately 100 nanometers (nm) to 500 micrometers (μm), wherein the particulate material has microporous, mesoporous or macroporous nature.
